(19) United States
(12) Patent Application Publication
(10) Pub. No.: US 2010/0099187 A1
(43) Pub. Date: Apr. 22, 2010

(54) METHOD FOR PRODUCING A VACCINE FOR THE TREATMENT OF CANCER

(75) Inventor: DAVID BERD, WYNCOTE, PA (US)

Correspondence Address: MORGAN, LEWIS & BOCKIUS LLP, 1701 MARKET STREET, PHILADELPHIA, PA, 19103-2921, US

(73) Assignee: AVAX TECHNOLOGIES, INC., PHILADELPHIA, PA (US)

(21) Appl. No.: 12/642,827

(22) Filed: Dec. 20, 2009

Related U.S. Application Data
(63) Continuation of application No. 11/331,468, filed on Jan. 13, 2006, now abandoned.
(60) Provisional application No. 60/644,364, filed on Jan. 14, 2005.
(60) Provisional application No. 60/696,951, filed on Jul. 6, 2005.

(30) Foreign Application Priority Data
Jan. 13, 2006 (US) PCT/US06/01115

(51) Int. Cl. C12N 5/02

(52) U.S. Cl. 435/374

(57) ABSTRACT

The present invention discloses a method for producing a haptenized vaccine from a tissue biopsy. The method includes obtaining a tissue biopsy, isolating the cells, irradiating the cells, haptenizing the cells, and cryopreserving the cells. The present invention also discloses a method for treating cancer using the vaccines produced by the methods described herein.

### STATEMENT OF RELATED APPLICATIONS

This application is a continuation of U.S. patent application Ser. No. 11/331,468, filed Jan. 13, 2006, which claims priority to U.S. Provisional Application No. 60/644,364, filed on Jan. 14, 2005, and U.S. Provisional Application No. 60/696,951, filed on Jul. 6, 2005, each of which are incorporated herein by reference in their entireties. This application is related to International Application No. PCT/US06/01115, filed Jan. 13, 2006, which is also incorporated herein by reference in its entirety.

### FIELD OF INVENTION

The present invention is directed to a method for producing sterile cancer vaccines. The vaccines comprise hapten-modified tumor cells and extracts and are useful for treatment of cancer by administering a therapeutically effective amount of a composition comprising a hapten-modified tumor cell or tumor cell extract to a patient in need of such treatment.

### BACKGROUND OF INVENTION

It was theorized in the 1960's that tumor cells bear specific antigens called tumor-specific antigens (“TSA”) which are not present on normal cells and that the immune response to these antigens might enable an individual to reject a tumor. It was later suggested that the immune response to TSA could be increased by introducing new immunological determinants on cells (Mitchison, Transplant. Proc., 1970, 2:92). Such a “helper determinant,” which, for example, can be a hapten, a protein, a viral coat antigen, a transplantation antigen, or a xenogenous cell antigen, could be introduced into a population of tumor cells. Clinically, the goal was to induce an immunologic reaction against the helper determinants, thereby increasing the amount of accompanying TSA, and destroying the tumor cells.

Fujiwara et al. (J. Immunol., 1984, 132:1571) demonstrated that certain haptenized tumor cells, i.e., tumor cells conjugated with the hapten trinitrophenyl (TNP), induced systemic immunity against unmodified tumor cells in a murine system, provided that the mice were first sensitized to the hapten in the absence of hapten-specific suppressor T cells. Flood et al. (J. Immunol., 1987, 138:3573) demonstrated that mice immunized with a TNP-conjugated, ultraviolet light-induced “regressor” tumor were able to reject a TNP-conjugated “progressor” tumor that was otherwise non-immunogenic. Moreover, these mice were subsequently resistant to challenge with unconjugated “progressor” tumor. In another experimental system, Fujiwara et al. (J. Immunol., 1984, 133:510) demonstrated that mice sensitized to trinitrochlorobenzene (TNCB) after cyclophosphamide pretreatment could be cured of large (10 mm) tumors by in situ haptenization of tumor cells; subsequently, these animals were specifically resistant to challenge with unconjugated tumor cells.

The existence of T cells that cross-react with unmodified tissues has recently been demonstrated. Class I MHC-restricted T cell clones generated from mice immunized with TNP-modified syngeneic lymphocytes respond to MHC-associated, TNP-modified “self” peptides (Ortmann, B., et al., J. Immunol., 1992, 148:1445). In addition, it has been established that immunization of mice with TNP-modified lymphocytes results in the development of splenic T cells that exhibit secondary proliferative and cytotoxic responses to TNP-modified cells in vitro (Shearer, G. M. Eur. J. Immunol., 1974, 4:527). The potential of lymphocytes elicited by immunization with DNP- or TNP-modified autologous cells to respond to unmodified autologous cells is of considerable interest because it may be relevant to two clinical problems: 1) drug-induced autoimmune disease, and 2) cancer immunotherapy. Regarding the former, it has been suggested that ingested drugs act as haptens, which combine with normal tissue protein forming immunogenic complexes that are recognized by T cells (Tsutsui, H., et al., J. Immunol., 1992, 149:706). Subsequently, autoimmune disease, e.g., systemic lupus erythematosus, may develop and continue even after withdrawal of the offending drug. This implies the eventual generation of T lymphocytes that cross-react with unmodified tissues.

Administration of cyclophosphamide, at high dose (1000 mg/M2) or low dose (300 mg/M2)[,] three days before sensitization with the primary antigen keyhole limpet hemocyanin markedly augments the acquisition of delayed type hypersensitivity to that antigen (Berd et al., Cancer Res., 1982, 42:4862; Cancer Res., 1984, 44:1275). Low dose cyclophosphamide pretreatment allows patients with metastatic melanoma to develop delayed type hypersensitivity to autologous melanoma cells in response to injection with autologous melanoma vaccine (Berd et al., Cancer Res., 1986, 46:2572; Cancer Invest., 1988, 6:335). Cyclophosphamide administration results in reduction of peripheral blood lymphocyte non-specific T suppressor function (Berd et al., Cancer Res., 1984, 44:5439; Cancer Res., 1987, 47:3317), possibly by depleting CD4+, CD45R+ suppressor inducer T cells (Berd et al., Cancer Res., 1988, 48:1671).

Conventional attempts to treat human cancer have been unsuccessful or only partially successful, and often have undesirable side effects. Attempts to treat cancer based on various immunological theories have also been unsuccessful.

### DETAILED DESCRIPTION

The present invention includes a method for preparing a vaccine from a tissue. In an embodiment of the invention, the method includes performing the following steps in the order listed:

1. obtaining a tissue sample;

2. extracting cells from the tissue sample;

3. irradiating the cells;

4. modifying the cells with a hapten; and

5. aliquoting the cells into vials.

In another embodiment, the method further includes a step of cryopreserving the cells after aliquoting them into vials.

In an embodiment of the invention, irradiating the cells makes the cells more immunogenic. In an embodiment of the invention, the haptenization process shuts down the metabolism of the cells, therefore, irradiation occurs prior to haptenization. Aliquoting the cells into single dose vials and, optionally, cryopreserving the cells as the last step allows for long storage times and for quality control of each batch of cells, which are commercially and regulatorily advantageous.

Tissue and Cell Types

The present invention is directed to preparing a vaccine from a tissue. The types of tissues from which a vaccine may be prepared include, without limitation, the following tissues: skin, blood, serum, saliva, sputum, urine, mucus, bone marrow, lymph, lung, liver, kidney, muscle, rectum, colon, breast, prostate, ovaries, testes, lymph nodes, or other tissues.

Obtaining a Tissue Sample

In an embodiment of the present invention, a tissue sample is isolated from a patient. In an embodiment of the present invention, a tissue sample is isolated through standard techniques known in the art, such as, taking a biopsy. In one embodiment, the tissue sample is obtained from a tumor. In another embodiment, the tissue sample is obtained by excising a tumor. In another embodiment, the tissue sample is a tumor. In an embodiment of the present invention, the tissue sample is a malignant or premalignant tumor. In another embodiment, a tissue sample is a solid or liquid tissue sample including, without limitation, all or part of a tumor, saliva, sputum, mucus, bone marrow, serum, blood, urine, lymph, or a tear from a patient suspected of having cancer.

In one embodiment, the tissue sample processed from the tumor is about 1 centimeter or greater in diameter. In another embodiment, the tissue sample processed from the tumor is about 1.5 centimeters or greater in diameter (about 1.8 grams). In yet another embodiment, the tissue sample processed from the tumor is about 1.8 centimeters or greater in diameter (about 3 grams). In another embodiment, the tumor tissue processed from the tumor is about 2 centimeters or greater in diameter (about 4.2 grams). In another embodiment, the tumor tissue is about 5 centimeters to about 10 centimeters or greater in diameter.

In an embodiment of the invention, a liquid tissue sample is about 0.1 milliliter or greater. In another embodiment, the liquid tissue sample is about 1 pint. In another embodiment, the liquid tissue sample is from about 10 milliliters to about 1 liter. In another embodiment, the liquid tissue sample is from about 100 milliliters to about 500 milliliters.

In an embodiment of the invention, the tissue sample obtained from a patient is large enough to produce the number of cells or cell equivalents needed to produce a vaccine of the present invention. In another embodiment, the tissue sample obtained from a patient is large enough to collect enough cells from the sample to begin an in vitro cell culture.

Extracting Cells

In an embodiment of the present invention, cells or cell equivalents are extracted from the tissue sample. In an embodiment of the present invention, the tissue is a tumor. In an embodiment of the present invention, the cells or cell equivalents are extracted from the tissue by mechanical dissociation. Mechanical dissociation includes, without limitation, cutting the tissue into small pieces, teasing the tissue, and/or forcing the tissue through a screen. In an embodiment of the invention, the cells are pelleted by centrifugation at from about 200 g to about 500 g for about 7 minutes to about 30 minutes. In an embodiment of the invention, the cells or cell equivalents are pelleted by centrifugation at about 300 g (about 1100 rpm) for about 7 minutes. In an embodiment of the invention, the supernatant is aspirated and the cells are resuspended in Hank's balanced salt solution (HBSS). In an embodiment of the invention, the HBSS includes human serum albumin (HSA). In an embodiment of the invention, the HSA is present in an amount of from about 0.05% to about 5%. In an embodiment of the invention, the HBSS includes a broad-spectrum antibiotic. In one embodiment, the broad-spectrum antibiotic is a fluoroquinolone. In another embodiment, the antibiotic is gentamycin. In another embodiment, the gentamycin is present in a concentration of about 50 micrograms per milliliter.

In one embodiment of the present invention, the cells or cell equivalents are isolated from the tissue using enzymatic digestion. In one embodiment, the enzyme used is collagenase, DNase, or a combination thereof.

In an embodiment of the invention, the tumor cells are stored and/or transported in a medium containing HBSS and a broad spectrum antibiotic. In one embodiment, the broad-spectrum antibiotic is a fluoroquinolone. In another embodiment, the broad spectrum antibiotic is gentamycin. In an embodiment of the invention, the percent (v/v) of HBSS is from about 95% to about 99.5% and the percent (v/v) of broad spectrum antibiotic is from about 0.5% to about 5%. In another embodiment, the percent (v/v) of HBSS is about 99.5% and the percent (v/v) of antiobiotic is about 0.5%.

Irradiation of Cells

In an embodiment of the present invention, the isolated cells are irradiated with gamma irradiation. A Gamacell 1000 Elite (MDS, Nordion, Ontario, Canada) is one example of the type of equipment required to gamma-irradiate the cells. In another embodiment, the cells are irradiated with x-rays. One example of how the cells may be irradiated by X-rays is through use of the X Ray machine Faxitron Model X-650. Other orthovoltage irradiators are also useful in the present invention. In an embodiment of the present invention, the isolated cells are irradiated at about 5-250 Grays (Gy). In another embodiment, the cells are irradiated at about 10-150 Gy. In another embodiment, the cells are irradiated at about 20-100 Grays. In yet another embodiment, the cells are irradiated at about 25 Gy (2500 cGy).

Without being bound to any particular theory, it is thought that irradiation of the tumor cells causes immunopotentiation, providing a boost to the immune system. In one embodiment, the cells are irradiated prior to haptenization.

Haptenization of Cells

In an embodiment of the present invention, the cells are haptenized. In one embodiment, the hapten may be dinitrophenyl (DNP) for example. Other haptens include, without limitation, trinitrophenyl (TNP), N-iodoacetyl-N′-(5-sulfonic 1-naphthyl)ethylene diamine, trinitrobenzenesulfonic acid, fluorescein isothiocyanate, arsenic acid benzene isothiocyanate, trinitrobenzenesulfonic acid, sulfanilic acid, arsanilic acid, dinitrobenzene-S-mustard. In an embodiment of the invention, a combination of haptens may also be used for conjugation to the tumor cell.

In one embodiment of the present invention, the haptenization process takes place in the absence of protein. In the presence of protein, DNP binds to the protein instead of the cells. In an embodiment of the invention, the cells are centrifuged to create a cell pellet. In another embodiment, the cells are resuspended in HBSS at a density in cells/ml required by the user. In another embodiment, the amount of dinitrofluorobenzene (DNFB) solution is calculated that is required to optimally haptenize the cells. In one embodiment, the DNFB added to a cell suspension is at a concentration of about 0.5 mM. In another embodiment, the cells and the DNFB are incubated for a period of about 10 to about 50 minutes at a temperature of about 15 to about 40 degrees Celsius. In another embodiment, the cells are centrifuged and resuspended in HBSS containing HSA. In one embodiment, the HSA is present in an amount of from about 0.05% to about 5%. In another embodiment, the HSA is present in an amount of about 1%. In another embodiment, the HSA is present in an amount of about 0.1%. In an embodiment of the invention, the haptenization process is as follows: The cells are pelleted by centrifugation at about 300 g (about 1100 RPM) for about 7 to about 12 minutes. HBSS (without protein or serum) is added to the cell pellet to bring the concentration of cells to about 5×10⁶ cells per milliliter. In an embodiment of the invention, for each 1.0 milliliter of cell suspension, about 0.1 milliliter of dinitrofluorobenzene (DNFB) solution (about 0.5 mM) is added. In an embodiment of the invention, this amount of DNFB can haptenize up to 10×10⁶ cells.

In one embodiment, the cell suspension is mixed and incubated at room temperature for 30 minutes, and gently mixed every 10 minutes. The cells are then washed twice in HBSS with HSA. In one embodiment, the HSA is present in any amount of from about 0.05% to about 5%. In an embodiment of the invention, the HSA absorbs any excess DNP.

In an embodiment of the present invention, hapten-modified cells are identified using flow cytometry. In another embodiment, hapten-modified cells are identified using ELISA or analysis of cell lysates by spectrophotometry, gas-liquid-chromatography, or mass spectroscopy.

Examples of haptenization media, methods for haptenization, and irradiation of cells are shown in U.S. application Ser. Nos. 08/203,004; 10/260,119; 10/025,195; U.S. Publication Nos. 2002-0009496; 2003-0068337; 2003-0170756; 2003-0165518; 2003-0064080; and U.S. Pat. Nos. 6,403,104; 6,458,369; 6,333,028, and 5,290,551, all of which are hereby incorporated by reference in their entirety.

In an embodiment of the invention, after the cells are haptenized, the cells are suspended in a freezing medium. In one embodiment, the freezing medium contains sucrose. In one embodiment, sucrose is present in an amount of from about 0% (w/v) to about 20% (w/v). In another embodiment, the sucrose is present in an amount of about 8% (w/v). In another embodiment, the freezing medium contains HSA. In one embodiment, a 25% (w/v) solution of HSA is present in the freezing medium in an amount of from about 0% (v/v) to about 50% (v/v). In another embodiment, a 25% (w/v) solution of HSA is present in the freezing medium in an amount of about 37% (v/v). In another embodiment, the remainder of the freezing medium is HBSS.

Aliquoting the Cells

In an embodiment of the present invention, the cells are aliquoted into single-dose vials. In one embodiment, the dosage of cells is at least 10⁴ tumor cells or cell equivalents. In another embodiment, the dosage of cells is at least 10⁵ cells or cell equivalents, and in another embodiment, the dosage of cells is at least 10⁶ cells or cell equivalents. In one embodiment, the dosage contains from about 10⁵ to about 2.5×10⁷ cells or cell equivalents, and in another embodiment, about 5×10⁵ cells or cell equivalents, in another embodiment, about 2.5×10⁶ cells or cell equivalents, and in another embodiment, about 5×10⁶ cells or cell equivalents. In another embodiment, the dosage contains up to about 7.5×10⁶ cells or cell equivalents. In another embodiment, the dosage contains up to about 20×10⁶ cells or cell equivalents.

Cryopreservation of Cells

In an embodiment of the present invention, the cells or cell equivalents are cryopreserved. In another embodiment, the cells or cell equivalents are used immediately after haptenization. In an embodiment of the present invention, the freezing medium includes HBSS with about 7-10% HSA and about 7-8% sucrose. In another embodiment, the freezing medium includes HBSS, about 7-10% HSA, and dimethylsulfoxide (DMSO). In one embodiment of the present invention, the cells or cell equivalents are stored in liquid nitrogen at −80 degrees Celsius. In another embodiment, the cells are stored in a −80 degrees Celsius freezer. In an embodiment of the present invention, the cells or cell equivalents are stored in a stepdown cryopreservation chamber. In an embodiment of the invention, the cells or cell equivalents are stable for up to 9 months at −80 degrees Celsius. In another embodiment, the cells or cell equivalents are stable for up to 6 months at −80 degrees Celsius.

Other Additives

In an embodiment of the present invention, other compositions may be co-administered with the vaccine upon thawing, prior to administration to a patient. For purposes of the present invention, co-administration includes administration together (i.e., simultaneously) and consecutively. These additives include, without limitation, adjuvants, cytokines, and pharmaceutically acceptable diluents.

In an embodiment of the invention, a vaccine is co-administered with an adjuvant. In one embodiment, the initial dose of the vaccine is not administered with an adjuvant. In another embodiment, the initial dose of the vaccine is administered with an adjuvant. Any known aqueous vehicle useful in drug delivery, such as and not limited to saline, may be used in accordance with the present invention as a carrier. In addition, any adjuvant known to skilled artisans may be useful in the delivery of the present invention. In an embodiment of the invention, the adjuvant has the property of augmenting an immune response to the tumor cell preparations of the present invention. Adjuvants useful in the invention include Bacille Calmette-Guerin (BCG), the synthetic adjuvant, QS-21 comprising a homogeneous saponin purified from the bark of Quillaja saponaria, Corynebacterium parvum (McCune et al., Cancer 1979 43:1619), saponins in general, detoxified endotoxin and cytokines such as IL-2, IL-4, gamma interferon (IFN-gamma), IL-12, IL-15, IL-27, GM-CSF and combinations thereof. In an embodiment of the invention, the adjuvant is BCG. In an embodiment of the invention, the adjuvant is administered with the vaccine at an amount of from about 0.1×10⁶ to about 20×10⁶ colony forming units (CFU).

In an embodiment of the invention, the cytokines useful in the invention include IL-2, IL-4, IL-12, IL-27, IFN-gamma, GM-CSF, and combinations thereof. In an embodiment of the present invention, the vaccine of the invention is used in conjunction with other cancer treatments including, without limitation, chemotherapy, radiation, antibodies, oligonucleotide sequences, and antisense oligonucleotide sequences.

In an embodiment of the present invention, the vaccine of the present invention is administered in a mixture with a pharmaceutically-acceptable carrier, selected with regard to the intended route of administration and the standard pharmaceutical practice. In an embodiment of the invention, dosages are set with regard to weight, and clinical condition of the patient. The proportional ratio of active ingredient to carrier naturally depends on the chemical nature, solubility, and stability of the compositions, as well as the dosage contemplated. In another embodiment of the invention, dosages are set with regard to the number of cells or cell equivalents administered in the vaccine.

The present invention also includes methods for treating cancer using the vaccines prepared by the method discussed above. The method includes administering the vaccine in an effective amount to a patient suffering from a tumor in need of such a vaccine.

Any malignant tumor may be treated according to the present invention including metastatic and primary cancers and solid and non-solid tumors. In one embodiment, solid tumors, include carcinomas, and non-solid tumors including hematologic malignancies are treatable with the vaccine of the present invention. In another embodiment, carcinomas treatable with the vaccine of the present invention include, without limitation, adenocarcinomas and epithelial carcinomas. In yet another embodiment, hematologic malignancies, including, without limitation, leukemias, lymphomas, and multiple myelomas are treatable with the vaccine of the present invention. The following are non-limiting examples of the cancers treatable with the vaccine prepared according to the method of the present invention: ovarian, advanced ovarian, leukemia, acute myelogenous leukemia, colon, colon metastasized to liver, rectal, colorectal, melanoma, breast, lung, kidney, and prostate cancers. In another embodiment, stage I, II, III, or IV cancers are treated according to the present invention. In another embodiment, stage III cancer is treated according to the method of the present invention. In yet another embodiment, stage IV cancer is treated according to the present invention. In another embodiment, a mammal suffering from a cancer is treated according to the present invention. In another embodiment, a human suffering from a cancer is treated according to the present invention.

In an embodiment of the present invention, the vaccine composition of the present invention is packaged in a dosage form suitable for intradermal, intravenous, intraperitoneal, intramuscular, or subcutaneous administration. In another embodiment, the dosage form may contain the vaccine of the invention to be reconstituted at the time of the administration with, for example, a suitable diluent.

In one embodiment of the present invention, the vaccine of the invention is administered by any suitable route, including inoculation and injection, for example, intradermal, intravenous, intraperitoneal, intramuscular, and subcutaneous. In an embodiment of the invention, one patient may have multiple sites of administration per each vaccine treatment. For example, the vaccine composition may be administered by intradermal injection into one, two, or three contiguous sites per administration. In one embodiment of the invention, the vaccine composition is administered on the upper arms or in the legs.

In an embodiment of the present invention, prior to administration of the vaccine composition of the invention, a patient is immunized to the hapten that is used to modify the tumor cells by applying the hapten to the skin. In one embodiment, dinitrofluorobenzene (DNFB) is used to immunize the patient. In one embodiment of the invention, the patient is not immunized to a hapten prior to vaccine administration.

In an embodiment of the present invention, the drug cyclophosphamide (CY) may be administered several days prior to each vaccine administration to augment the immune response to the tumor cells. In another embodiment, CY is administered only prior to the first vaccine administration.

In an embodiment of the present invention, the vaccination protocol is as follows:

| | Dose | | |
|---|---|---|---|
| Day | No. | Drug | Dose |
| 1 | 1 | Vaccine Only | cells only* |
| 7 | | Cyclophosphamide | 300 mg/m² |
| 10 | 2 | Vaccine BCG | cells plus BCG at 1-8 × 10⁶ CFU |
| 17 | 3 | Vaccine BCG | cells plus BCG at 1-8 × 10⁶ CFU |
| 24 | 4 | Vaccine BCG | cells plus BCG at 1-8 × 10⁵ CFU |
| 31 | 5 | Vaccine BCG | cells plus BCG at 1-8 × 10⁵ CFU |
| 38 | 6 | Vaccine BCG | cells plus BCG at 1-8 × 10⁴ CFU |
| 45 | 7 | Vaccine BCG | cells plus BCG at 1-8 × 10⁴ CFU |
| 6 | 8 | Vaccine BCG | cells plus BCG at 1-8 × 10⁴ CFU |
| month | | | |
| booster | | | |

*Cells will be at a dose of either 5 × 10⁵, 2.5 × 10⁶, or 5 × 10⁶ per dose.

### EXAMPLES

### Example 1

### Preparation of Vaccine

Primary non-small cell lung cancer (NSCLC) tumors were obtained from 19 patients undergoing surgery (10 adenocarcinoma; 8 squamous cell carcinoma; 1 large cell carcinoma). Sixteen of the tumors were in stage I, one tumor was in stage II, and two tumors were in stage IIIA. Tumors were surgically resected in a standard manner, and a portion of each tumor was excised. The excised portion was transported to AVAX Technologies, Inc. (Lyon, France) in a sterile container at 4 degrees Celsius.

The vaccines were prepared at AVAX using the method of the present invention. Tumor cells were extracted by mechanical dissociation. The biopsy was washed with 50 milliliters of Hank's Buffered Salt Solution (HBSS). The biopsy was cut into small pieces and placed into 10 milliliters of HBSS. The pieces were transferred to NETWELL strainers with 3 milliliters HBSS/human serum albumin (HSA)/gentamycin per well. The HSA concentration is from about 0.1% to about 1%, and the gentamycin concentration is about 50 micrograms per milliliter. The cells were then pressed against the strainer using a sterile syringe plunger. The strainers were washed twice with 2 milliliters of the HBSS/HSA/gentamycin solution. The cells were recovered, and the volume of the cell suspension was adjusted to 30 milliliters with HBSS.

The tumor cells were then irradiated using an X Ray machine Faxitron Model X-650, at 2500 cGy. Following irradiation, the tumor cells were centrifuged at about 300 g for about 7 minutes and washed twice with HBSS. The volume and cell concentration was adjusted to 25×10⁶ cells per milliliter using HBSS. Two milliliters of the cell suspension was removed and transferred into a separate tube for DTH doses, and stored at 4 degrees Celsius.

The cells were modified with the hapten DNP. One volume of haptenization media per 10 volumes of cell suspension was added to the cell suspension, and incubated for 30 minutes at room temperature. The haptenization media included a dinitrofluorobenzene (DNFB) solution at about 0.5 mM. The cell suspension was then centrifuged at about 300 g for about 7 minutes. The cells were washed twice with HBSS/1% HSA. The cells were re-suspended in freezing media containing HBSS with about 7-10% HSA and about 7-8% sucrose, at a concentration of 25×10⁶ cells per milliliter. Approximately 250 microliters of the cell suspension (about 5.0×10⁶ cells) was added to each cryotube and the cryotubes were placed in the −80 degrees Celsius freezer.

The vaccines were then measured for sufficient quantity for administration in a clinical trial, and lymphocyte contamination.

Sufficient quantities of DNP-modified vaccine were produced from 13 out of 19 tumors. Where at least 3 grams of tumor tissue was obtained (about 1.8 cm in diameter), 100% of the vaccines manufactured were sufficient for administering in a clinical trial. Flow cytometry analysis revealed that all vaccines were DNP-modified, and that lymphocyte contamination (median of 20%, range from 3-37%) in the vaccines was lower than previously observed in DNP-vaccines produced from melanoma lymph node metastases. All NSCLC vaccines were free of aerobic and anaerobic bacteria in a standard 14-day sterility assay. No endotoxin was detectable in any of the vaccine samples.

This example demonstrates that vaccines generated from NSCLC tumors are very clean and sterile, and do not present any bioburden issues.

### Example 2

### Treatment of Lung Cancer with Vaccine

Three patient groups, A, B, and C are tested to determine the effectiveness of a DNP-modified vaccine to treat cancer. Group A receives a dose of 5×10⁵ cells per vaccination, Group B receives a dose of 2.5×10⁶ cells per vaccination, and Group C receives a dose of 5×10⁶ cells per vaccination. Each patient is tested for DTH approximately 14 days prior to Dose 1 on the dosing chart below. DTH testing is repeated about 2½ weeks after dose No. 6. After the DTH readings, each patient follows the dosing schedule set forth below. Clinical assessments of each patient are conducted.

| | Dose | | |
|---|---|---|---|
| Day | No. | Drug | Dose |
| 1 | 1 | Vaccine Only | cells only* |
| 7 | | Cyclophosphamide | 300 mg/m² |
| 10 | 2 | Vaccine BCG | cells plus BCG at 1-8 × 10⁶ CFU |
| 17 | 3 | Vaccine BCG | cells plus BCG at 1-8 × 10⁶ CFU |
| 24 | 4 | Vaccine BCG | cells plus BCG at 1-8 × 10⁵ CFU |
| 31 | 5 | Vaccine BCG | cells plus BCG at 1-8 × 10⁵ CFU |
| 38 | 6 | Vaccine BCG | cells plus BCG at 1-8 × 10⁴ CFU |
| 45 | 7 | Vaccine BCG | cells plus BCG at 1-8 × 10⁴ CFU |
| 6 | 8 | Vaccine BCG | cells plus BCG at 1-8 × 10⁴ CFU |
| month | | | |
| booster | | | |

*Cells will be at a dose of either 5 × 10⁵, 2.5 × 10⁶, or 5 × 10⁶ cells per dose.

Post-vaccine DTH tests to autologous cancer cells, both DNP-modified and unmodified are conducted to determine sensitivity to cells. Secondary endpoints, such as relapse-free survival and overall survival, are measured to determine the effectiveness of the vaccine.

It will be apparent to those skilled in the art that various modifications and variations can be made in the device of the present invention without departing from the scope of the invention. Thus, it is intended that the present invention cover the modifications and variations of the present invention provided they come within the scope of the appended claims and their equivalents.

What is claimed is:

1. A method for preparing haptenized lung cancer tumor cells comprising:
mechanically dissociating lung cancer tumor cells from a tissue sample;
pelleting said mechanically dissociated lung cancer tumor cells;
suspending said pelleted lung cancer tumor cells in a re-suspension solution including a broad-spectrum antibiotic;
haptenizing said lung cancer tumor cells; and
suspending said haptenized lung cancer tumor cells in a freezing medium.

2. The method of claim 1, wherein said lung cancer tumor cells are primary non-small cell lung carcinoma tumor cells.

3. The method of claim 1, further comprising irradiating said lung cancer tumor cells prior to haptenizing said lung cancer tumor cells.

4. The method of claim 1, further comprising freezing said haptenized lung cancer tumor cells suspended in the freezing medium.

5. The method of claim 4, wherein freezing includes storing said haptenized lung cancer tumor cells suspended in the freezing medium at about −80 degrees C.

6. The method of claim 1, wherein said freezing medium comprises from about 7 to about 10 percent HSA, and from about 7 to about 8 percent sucrose.

7. The method of claim 1, wherein said re-suspension solution further includes from about 0.05 percent HSA to about 5 percent HSA.

8. The method of claim 1, wherein said broad spectrum antibiotic is gentamycin.

9. The method of claim 1, wherein said broad spectrum antibiotic is a fluoroquinolone.

10. The method of claim 1, wherein haptenizing said lung cancer tumor cells includes:
incubating said lung cancer tumor cells in a dinitrofluorobenzene solution without protein or serum.

11. The method of claim 10, further comprising washing said lung cancer tumor cells with a wash solution including HSA after incubating said lung cancer tumor cells in the dinitrofluorobenzene solution.

12. The method of claim 1, further comprising analyzing said haptenized lung cancer tumor cells using flow cytometry.

13. A method for preparing haptenized lung cancer tumor cells comprising:
obtaining at least a portion of a stage I, stage II, or stage IIIA primary non-small cell lung cancer tumor from a patient;
extracting tumor cells from said tumor portion, wherein extracting said tumor cells consists of mechanically dissociating said tumor cells from said tumor portion;
irradiating said mechanically dissociated tumor cells;
haptenizing said irradiated tumor cells; and
suspending said haptenized tumor cells in a freezing medium.

14. The method of claim 13, further comprising freezing said haptenized tumor cells suspended in the freezing medium.

15. The method of claim 13, wherein haptenizing said tumor cells consists of DNP-modification of said tumor cells.

16. The method of claim 13, wherein haptenizing said tumor cells includes:
incubating said tumor cells in a dinitrofluorobenzene solution without protein or serum.

17. The method of claim 16, further comprising washing said tumor cells with a wash solution including HSA after incubating said tumor cells in the dinitrofluorobenzene solution.

18. The method of claim 13, wherein said freezing medium comprises HSA and sucrose.

19. The method of claim 13, further comprising analyzing said haptenized tumor cells using flow cytometry.

20. A method for preparing hapten-modified lung cancer tumor cells comprising:
obtaining at least a portion of a primary non-small cell lung cancer tumor from a patient;
extracting tumor cells from said tumor portion by mechanical dissociation;
suspending said mechanically dissociated tumor cells in a solution including serum albumin and a broad-spectrum antibiotic;
irradiating said mechanically dissociated tumor cells;
incubating said irradiated tumor cells in a dinitrofluorobenzene solution without protein or serum;
washing said incubated tumor cells with a solution including serum albumin;
storing said washed tumor cells in a solution including serum albumin and sucrose; and
identifying hapten-modified tumor cells from a population of said stored tumor cells.
